(19) United States
(12) Patent Application Publication
(10) Pub. No.: US 2022/0032769 A1
(43) Pub. Date: Feb. 3, 2022

(54) Vehicle Control System With Impaired Driver Prevention And Biometric Detection

(71) Applicant: Randall Hicks, Canton, OH (US)

(72) Inventor: Randall Hicks, Canton, OH (US)

(21) Appl. No.: 17/388,902

(22) Filed: Jul. 29, 2021

Related U.S. Application Data
(60) Provisional application No. 63/057,936, filed on Jul. 29, 2020.

(51) Int. Cl. B60K 28/06; B60R 16/023; A61B 5/08; A61B 5/097; A61B 5/00; G01N 33/66

(52) U.S. Cl. CPC B60K 28/063; B60R 16/0231; A61B 5/082; A61B 5/097; A61B 2560/0443; A61B 5/6893; G01N 33/66; A61B 5/0077; A61B 5/4845

(57) ABSTRACT

A vehicle control system with impaired driver prevention and biometric detection includes a vehicle with an ignition interlock circuit and one or more vehicle electrical systems, a detection device with an internal sensor system, and a mobile device having and one or more biometric sensors. The system operates by initiating a vehicle start command via the mobile device, prompting a user for a scan, receiving the scan from the detection device, whereby the scan includes at least a blood alcohol content percentage of the user, acquiring a sensed biometric image via the biometric sensors of the mobile device, and comparing the sensed biometric image to one or more stored biometric images. If the biometric image does not match a stored biometric image, or if the scan includes a blood alcohol content above a threshold value, then the vehicle start command is canceled.

### CROSS REFERENCE TO RELATED APPLICATIONS

This application claims the benefit of U.S. Provisional Application No. 63/057,936 filed on Jul. 29, 2020. The above identified patent application is herein incorporated by reference in its entirety to provide continuity of disclosure.

### BACKGROUND OF THE INVENTION

The present invention relates to improvements in vehicle safety systems. More specifically, the present invention provides a Vehicle Control System With Impaired Driver Prevention And Biometric Detection that is configured to prevent an individual from starting a vehicle ignition if the individual's blood alcohol level or other parameter is detected to be above a threshold value and if a biometric scan of the individual does not match a stored biometric image.

Many individuals are affected by the actions of impaired drivers. Driving a vehicle while drunk or otherwise impaired can lead to significant danger of serious vehicular accidents, often resulting in serious injury or death. Unfortunately, preventing impaired driving can be a difficult task, as without previously mandated vehicle modification, such as breathalyzer interrupts, any driver has the potential to become an impaired driver. Law enforcement officials often identify impaired driving and prevent accidents before they occur, but this activity fails to properly prevent impaired driving before it occurs. Unfortunately, law enforcement can be too late to prevent serious accidents leading to injury and death. There is further risk of individuals accessing vehicles that they are unauthorized to access, whether it be for theft or other nefarious purposes. Therefore, a system that can prevent users from operating a vehicle in an impaired state, while also providing remote control of various vehicle systems and restricting access to the vehicle to approved biometric images, is desired.

There exist devices in the known art that relate to ignition interlock systems that can only engage the ignition of the vehicle when a breathalyzer or similar device detects a blood alcohol content (BAC) below a certain threshold percentage. However, these devices have several drawbacks. For example, these devices are typically hardwired in a single vehicle and cannot be utilized with other vehicles. Further, the hardwiring limits the range and effectives of such devices, meaning some individuals will choose not to use them. Further, the devices in the known art fail to include identification mechanisms such as biometric scanning and image matching to prevent an unauthorized user from gaining access to the vehicle. In light of the devices disclosed in the known art, it is submitted that the present invention substantially diverges in design elements from the known art and consequently it is clear that there is a need in the art for an improvement to existing vehicle control systems and ignition interlock systems. In this regard the present invention substantially fulfills these needs.

### SUMMARY OF THE INVENTION

In view of the foregoing disadvantages inherent in the known types of vehicle control and ignition interlock systems now present in the known art, the present invention provides a Vehicle Control System With Impaired Driver Prevention And Biometric Detection wherein the same can be utilized for providing convenience and safety for the user when traveling in a vehicle.

In an exemplary embodiment vehicle control system includes a vehicle having a wireless transceiver operably connected to a vehicle battery, an ignition interlock circuit, and one or more vehicle electrical systems, a detection device having a housing having an external mouthpiece operably connected to an internal sensor system, which is operably connected to a battery and a wireless transceiver, and a mobile device having a housing with a display, an input mechanism, a wireless transceiver, and one or more biometric sensors operably connected to a battery.

The mobile device further includes a processer, a non-transitory computer readable medium operatively connected to the processor, and a logic stored in the non-transitory computer readable medium that, when executed by the processor, causes the remote vehicle access system to perform a method. The method includes initiating a vehicle start command via the input mechanism of the mobile device, prompting a user for a scan via the display of the mobile device, receiving, at the mobile device, a scan from the detection device, whereby the scan includes at least a blood alcohol content percentage of the user, acquiring a sensed biometric image via the one or more biometric sensors of the mobile device, and comparing the sensed biometric image to one or more stored biometric images. If the biometric image does not match a stored biometric image, then the system cancels the vehicle start command. If the scan includes a blood alcohol content percentage above a threshold value, then the system cancels the vehicle start command. If the scan includes a blood alcohol content percentage below a threshold value, and if the biometric data matches a stored biometric image, then the vehicle start command is transmitted to the vehicle wireless transceiver, thus deactivating the ignition interlock circuit and starting the vehicle ignition.

One object of the present invention is to provide a Vehicle Control System With Impaired Driver Prevention And Biometric Detection that prevents impaired drivers from using the vehicle and also prevents unauthorized individuals from using the vehicle.

Another object of the present invention is to provide a Vehicle Control System With Impaired Driver Prevention And Biometric Detection that includes a detection device having a slot for a test strip to test other parameters such as blood sugar levels, for example.

An additional object of the present invention is to a Vehicle Control System With Impaired Driver Prevention And Biometric Detection that can be either hardwired into a single vehicle or an auxiliary system that can be installed in different vehicles.

A further object of the present invention is to provide a Vehicle Control System With Impaired Driver Prevention And Biometric Detection that can automatically contact an emergency authority if a blood alcohol content above a particular level is detected by the detection device.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of a detection device of an embodiment of the Vehicle Control System With Impaired Driver Prevention And Biometric Detection.

FIG. 2 shows a diagram of the components of the Vehicle Control System With Impaired Driver Prevention And Biometric Detection.

### DETAILED DESCRIPTION OF THE INVENTION

Reference is made herein to the attached drawings. Like reference numerals are used throughout the drawings to depict like or similar elements of the seatbelt ignition interlock system. For the purposes of presenting a brief and clear description of the present invention, the preferred embodiment will be discussed as used for preventing a vehicle from starting when an individual has a blood alcohol content above a threshold level or when a biometric scan of the individual fails to identify them as an authorized user of the vehicle. The figures are intended for representative purposes only and should not be considered to be limiting in any respect.

According to some embodiments, the operations, techniques, and/or components described herein can be implemented as (i) a special-purpose computing device having specialized hardware and a logic hardwired into the computing device to persistently perform the disclosed operations and/or techniques or (ii) a logic that is implementable on an electronic device having a general purpose hardware processor to execute the logic and a computer-readable medium, e.g. a memory, wherein implementation of the logic by the processor on the electronic device provides the electronic device with the function of a special-purpose computing device.

In the interests of economy, the present disclosure refers to “a computer-readable medium,” “a processor,” and so on. However, this should not be read as limiting in any way as the present disclosure contemplates embodiments of the present invention utilizing “one or more computer-readable media,” “one or more processors,” and so on. Unless specifically limited to a single unit, “a” is intended to be equivalent to “one or more” throughout the present disclosure.

According to some embodiments, the operations, techniques, and/or components described herein can be implemented by an electronic device, which can include one or more special-purpose computing devices. The special-purpose computing devices can be hard-wired to perform the operations, techniques, and/or components described herein, or can include digital electronic devices such as one or more application-specific integrated circuits (ASICs) or field programmable gate arrays (FPGAs) that are persistently programmed to perform the operations, techniques and/or components described herein, or can include one or more general purpose hardware processors programmed to perform such features of the present disclosure pursuant to program instructions in firmware, memory, other storage, or a combination. Such special-purpose computing devices can also combine custom hard-wired logic, ASICs, or FPGAs with custom programming to accomplish the technique and other features of the present disclosure. The special-purpose computing devices can be desktop computer systems, portable computer systems, handheld devices, networking devices, or any other device that incorporates hard-wired and/or program logic to implement the techniques and other features of the present disclosure.

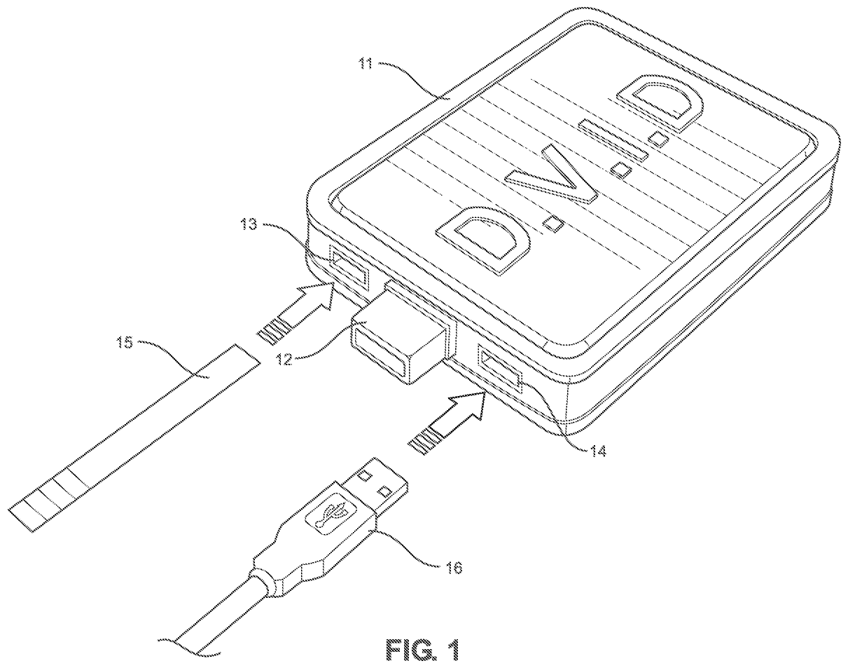

Referring now to FIG. 1, there is shown a perspective view of a detection device of an embodiment of the Vehicle Control System With Impaired Driver Prevention And Biometric Detection. The system requires the detection device to be utilized before allowing the vehicle ignition to start. The detection device includes a housing 11 having an external mouthpiece 12. The external mouthpiece 12 is operably connected to an internal sensor system, which is operably connected to a battery and a wireless transceiver. The external mouthpiece is utilized by the user, whereby the user's exhaled breath travels into the internal sensor system. The internal sensor system includes at least a blood alcohol content (BAC) sensor configured to detect the BAC of the user from their exhalations.

In some embodiments, the detection device housing 11 further includes an external slot 13 that can receive a test strip 15. The slot 13 extends inwardly to reach the internal sensor system, such that the material collection end of the test strip 15 can be inserted into the internal sensor system. In such an embodiment, the internal sensor system can include additional detection systems and sensors, such as a mechanism for detecting the blood sugar level of the individual, for example. A test strip 15 including a blood sample on its tip can be inserted into the device housing 11 through the slot 13. Since low blood sugar can be a sign of an impending impairment, the system can be configured such that a low blood sugar being detected will prevent the vehicle ignition from starting the vehicle engine. Further, in the shown embodiment, the housing 11 includes a port 14 that removably receives a USB connector 16. This can be utilized to provide power to charge the device's internal battery, or can also be utilized as a hardwired data transfer connection to transmit data detected by the internal sensor system to some external device, such as the mobile device of the present system.

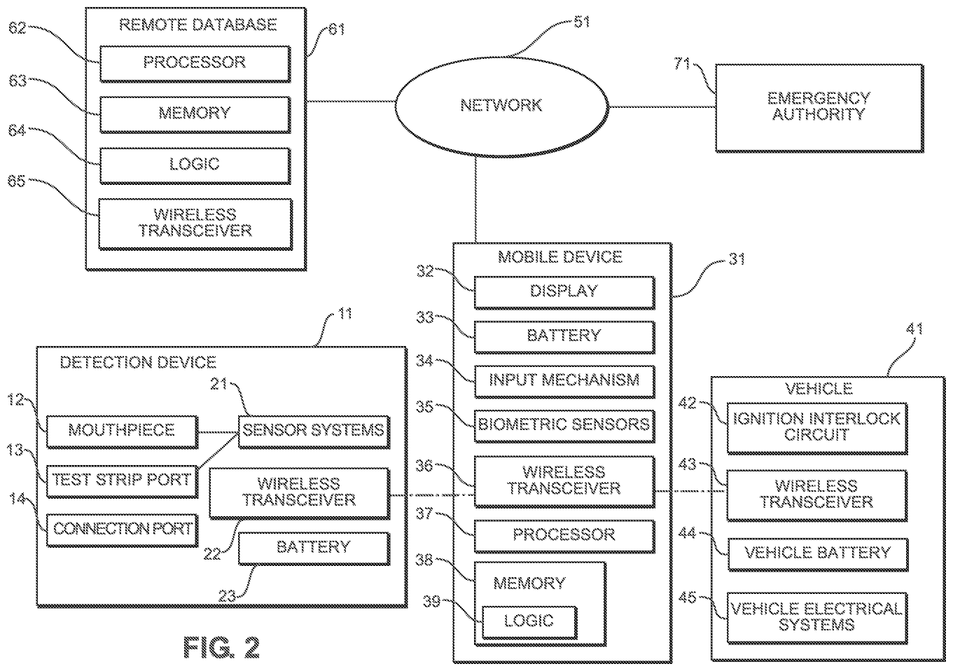

Referring now to FIG. 2, there is shown a diagram of the components of the Vehicle Control System With Impaired Driver Prevention And Biometric Detection. The system generally includes a mobile device 31, a detection device 11, and a vehicle 41. The mobile device 31 is utilized to control the system and to complete the initial setup of the system. During setup, the user registers the vehicle with the mobile device via inputting the vehicle identification number. This creates a user profile associated with their vehicle's vehicle identification number (VIN), both of which are stored on a remote database 61, such that the user's system functions only with their authorized vehicles.

The vehicle 41 includes an ignition interlock circuit 42 configured to only allow operation of the vehicle ignition if certain parameters are met. The vehicle 41 further includes a wireless transceiver 43, and one or more other vehicle electrical systems 45 powered by the vehicle battery 44. In some embodiments, the ignition interlock circuit 42 and wireless transceiver 43 are integrated into the one or more vehicle electrical systems 45 via hardwired connections made during the vehicle manufacturing process. In other embodiments, the wireless transceiver 43 and ignition interlock circuit 42 of the vehicle 41 are removably secured within the vehicle 41, such that the system can be utilized with different vehicles. Since the vehicle electrical systems 45 are operably connected to the wireless transceiver 43, and due to the mobile device's wireless transceiver 36 being in wireless communication with the vehicle wireless transceiver 43, the system is configured to facilitate controlling the vehicle electrical systems 45 via the mobile device 31. Some example vehicle electrical system 45 that can be remotely controlled include vehicle windows, lights, air conditioning, radio, and the like.

The detection device 11 includes a mouthpiece 12 and, in some embodiments, a test strip port 13 operably connected to the internal sensor systems 21. The connection port 14 is utilized to charge the battery and/or transfer data, along with the wireless transceiver 22. The mobile device 31 includes a display 32, an input mechanism 34 such as a touchscreen for example, one or more biometric sensors 35, such as a camera sensor for example, a wireless transceiver 36, a processor 37, a memory 38, and a logic 39 stored on the memory 38. when executed by the processor, causes the remote vehicle access system to perform a method. The method includes initiating a vehicle start command via the input mechanism 34 of the mobile device 31 and prompting a user for a scan via the display 32 of the mobile device 31, which can display a software application that facilitates the user interaction with the mobile device 31. The scan is done with the detection device 11, which can be utilized to detect the user's BAC level, blood sugar level, and other parameters that tend to indicate impairment if above or below a certain value.

The mobile device 31 then receives a scan from the detection device 11, whereby the scan includes at least a blood alcohol content percentage of the user. The mobile device 31 is then utilized to sense a biometric image via the one or more biometric sensors. In one embodiment, the biometric image includes a fingerprint scan. In another embodiment, the biometric image includes a retinal scan. In yet another embodiment, the biometric image includes a facial recognition image scan. The sensed biometric images are compared to one or more stored biometric images. The stored biometric images are provided by the initial authorized user during the initial setup of the system. In some embodiments, the stored biometric images are stored on the memory 38 of the mobile device. In other embodiments, the stored biometric images are stored on a remote database 61, which may include its own processor 62, memory 63, logic 64, and wireless transceiver 65. In such an embodiment, the wireless transceiver 36 of the mobile device 31 is in wireless communication with the remote database 61 via a network 51, such as the internet, for example. During initial setup, the user can store the approved biometric images, whether they be fingerprints, retinal scans, facial recognition scans, or the like. This allows the mobile device 31 to retrieve the stored images to compare them with the scanned images created by the mobile device's 31 biometric sensors 35.

If the biometric image does not match a stored biometric image, then the system automatically cancels the vehicle start command, and the ignition interlock circuit 42 prevents the vehicle engine from otherwise being started. If the scan includes a blood alcohol content percentage above a threshold value, then the system automatically cancels the vehicle start command. If the scan includes a blood alcohol content percentage below the threshold value, and if the biometric data matches a stored biometric image, then the system transmits the vehicle start command to the vehicle wireless transceiver 43, thus deactivating the ignition interlock circuit 41 and starting the vehicle ignition.

The present invention therefore prevents impaired and unauthorized individuals from accessing a vehicle. In some embodiments, the system is further configured to transmit, via the wireless transceiver 36 of the mobile phone 31, a communication to an emergency authority 71 via the network 51 if the scan includes a blood alcohol content percentage above a threshold value. In this way, law enforcement can be alerted and respond to the location of the impaired individual.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A vehicle control system, comprising:
a vehicle comprising a wireless transceiver operably connected to a vehicle battery, an ignition interlock circuit, and one or more vehicle electrical systems;
a detection device comprising a housing having an external mouthpiece operably connected to an internal sensor system, which is operably connected to a battery and a wireless transceiver;
a mobile device comprising a housing having a display, an input mechanism, a wireless transceiver, and one or more biometric sensors operably connected to a battery;
the mobile device further comprising a processer, a non-transitory computer readable medium operatively connected to the processor, and a logic stored in the non-transitory computer readable medium that, when executed by the processor, causes the remote vehicle access system to perform a method, the method comprising:
initiating a vehicle start command via the input mechanism of the mobile device;
prompting a user for a scan via the display of the mobile device;
receiving, at the mobile device, a scan from the detection device, whereby the scan includes at least a blood alcohol content percentage of the user;
acquiring a sensed biometric image via the one or more biometric sensors of the mobile device;
comparing the sensed biometric image to one or more stored biometric images;
if the biometric image does not match a stored biometric image, then cancelling the vehicle start command;
if the scan includes a blood alcohol content percentage above a threshold value, then cancelling the vehicle start command;
if the scan includes a blood alcohol content percentage below a threshold value, and if the biometric data matches a stored biometric image, then transmitting the vehicle start command to the vehicle wireless transceiver, thus deactivating the ignition interlock circuit and starting the vehicle ignition.

2) The vehicle control system of claim 1, wherein the method further comprises registering the vehicle with the mobile device via inputting the vehicle identification number.

3) The vehicle control system of claim 1, wherein the method further comprises transmitting, via the wireless transceiver of the mobile device, a communication to an emergency authority if the scan includes a blood alcohol content percentage above a threshold value.

4) The vehicle control system of claim 1, wherein the scan further includes blood sugar level data.

5) The vehicle control system of claim 1, wherein the housing of the detection device further comprises a test strip port operably connected to the sensor system, such that the sensor system is configured to detect properties of a test strip inserted into the test strip port.

6) The vehicle control system of claim 1, wherein the one or more stored biometric images are stored on the memory of the mobile device.

7) The vehicle control system of claim 1, wherein the one or more stored biometric images are stored in a remote database that is accessible by the mobile device via a wireless network.

8) The vehicle control system of claim 5, wherein the scan includes blood sugar data as determined by test strip inserted into the test strip port.

9) The vehicle control system of claim 1, wherein the housing of the detection device further comprises an external port configured to charge the internal battery.

10) The vehicle control system of claim 7, wherein the external port is further configured to exchange data via a wired connection with the mobile device.

11) The vehicle control system of claim 1, wherein the wireless transceiver of the vehicle is integrated into the one or more vehicle electrical systems.

12) The vehicle control system of claim 1, wherein the wireless transceiver of the vehicle is removably secured within the vehicle, such that the wireless transceiver can be utilized with different vehicles.

13) The vehicle control system of claim 1, wherein the sensed biometric image includes a fingerprint image.

14) The vehicle control system of claim 1, wherein the sensed biometric image includes a facial recognition image.

15) The vehicle control system of claim 1, wherein the sensed biometric image includes a retinal scan.

16) The vehicle control system of claim 1, wherein the mobile device wireless transceiver being in wireless communication with the vehicle wireless transceiver is configured to facilitate controlling the vehicle electrical systems via the mobile device.
